(19) United States
(12) Patent Application Publication
(10) Pub. No.: US 2010/0100007 A1
(43) Pub. Date: Apr. 22, 2010

(54) BLOOD TESTING DEVICE

(76) Inventor: Yoko SAKAMOTO, Ehime (JP)

Correspondence Address: WENDEROTH, LIND & PONACK L.L.P., 1030 15th Street, N.W., Suite 400 East, Washington, DC, 20005-1503, US

(21) Appl. No.: 12/579,658

(22) Filed: Oct. 15, 2009

(30) Foreign Application Priority Data
Oct. 21, 2008 (JP) 2008-270585

(51) Int. Cl. A61B 5/151

(52) U.S. Cl. 600/576

(57) ABSTRACT

A blood testing device includes (i) a main body, having a puncture component for puncturing a finger, a finger contact component where the finger to be punctured is placed during puncture by the puncture component, a sensor receptacle in which a blood sensor can be mounted, and a measurement component that measures concentration of a component in the blood held in the blood sensor, and (ii) at least one main body support that has a first space into which a finger other than the finger to be punctured is inserted, and that is provided to the main body directly or via another member.

### CROSS-REFERENCE TO RELATED APPLICATIONS

This application claims priority under 35 U.S.C. §119 to Japanese Patent Application No. 2008-270585 filed on Oct. 21, 2008. The entire disclosure of Japanese Patent Application No. 2008-270585 is hereby incorporated herein by reference.

### BACKGROUND

1. Technical Field

The present invention relates to a blood testing device.

2. Background Art

As shown in FIGS. 25 and 26, a conventional blood testing device 1 includes an insertion hole 4, a cartridge 5, a display component 6, and a laser emission device (not shown).

A finger 3a that is to be punctured is inserted into the insertion hole 4. The cartridge 5 is provided so as to come into contact with the finger inserted in the insertion hole 4. The cartridge 5 is equipped with a sensor (not shown) and a holder (not shown) in which the sensor is mounted. The cartridge 5 is disposed at the front of the laser emission device (on the finger side).

As shown in FIG. 26, the user grasps the blood testing device 1 with the left hand 3. The finger 3a (index finger) is inserted in the hole 4 at this time. A puncture button (not shown) is then pressed with the thumb, for example. This causes the laser emission device to emit a laser beam. The laser beam irradiates (punctures) the finger 3a, and blood seeps out of the punctured finger 3a. This blood is captured by the sensor. The sensor electrochemically converts the glucose concentration in the blood into an electrical signal. This electrical signal is converted into a digital signal and then transmitted to a computer (not shown) of the blood testing device 1. The computer converts the digital signal into a blood sugar value. The calculated blood sugar value is displayed on the display component 6.

Prior art such as this is discussed in Japanese Laid-Open Patent Application 2008-67743, for example.

### SUMMARY

A blood testing device according to an aspect of the present invention includes a main body including a puncture component configured to puncture a finger, a finger contact component arranged to contact a finger to be punctured during puncture by the puncture component, a sensor receptacle arranged to be mounted with a blood sensor, and a measurement component that measures a concentration of a component in the blood held in the blood sensor; and at least one main body support that has a first space into which a finger other than the finger to be punctured is inserted, and that is provided to the main body directly or via another member.

### BRIEF DESCRIPTION OF DRAWINGS

Referring now to the attached drawings, which form a part of this original disclosure:

FIG. 1 is an oblique view of the blood testing device in a first embodiment;

FIGS. 2A to 2C are plan views of the blood testing device when the cap has been slid downward, with FIG. 2A being a front view, FIG. 2B a side view (in FIG. 2B, the left side is the front face side, and the right side is the rear face side), and FIG. 2C a rear view (for all of the drawings referred to in this Specification, unless otherwise specified, “front face” means the face of the blood testing device on which the display component is provided, and “rear face” means the face on the opposite side);

FIGS. 3A to 3C are plan views of the blood testing device when the cap has been slid upward, with FIG. 3A being a front view, FIG. 3B a side view (in FIG. 3B, the left side is the front face side, and the right side is the rear face side), and FIG. 3C a rear view;

FIGS. 4A and 4B are plan views of the main body support of the blood testing device, with FIG. 4A being a view of the main body support in the same direction as in FIG. 3C, and FIG. 4B a view of the side face (in FIG. 4B, the left side is the front face side, and the right side is the rear face side);

FIG. 5 is a plan view of the main body of the blood testing device from the rear face side;

FIG. 6 is a plan view of the blood testing device from the rear face side;

FIGS. 7A and 7B are diagrams illustrating the adjustment of the distance between the puncture hole and the support hole in the blood testing device;

FIG. 8 is an oblique view of the usage state of the blood testing device;

FIG. 9A is a diagram of the constitution around the finger contact component, and FIG. 9B is a cross section that is perpendicular to the plane of view in FIG. 9A and parallel to the vertical direction in FIG. 9A;

FIG. 10 is a cross section of the main body of the blood testing device;

FIG. 11 is a cross section of the blood sensor mounted in the blood testing device;

FIG. 12 is a plan view of the internal configuration of the blood sensor;

FIG. 13 is an oblique view of the outside of the blood sensor;

FIG. 14 is a block diagram of a laser unit and a high voltage generating circuit;

FIG. 15 is a block diagram of the constitution of the measurement circuit and its surroundings;

FIG. 16 is a flowchart of the measurement operation with the blood testing device;

FIGS. 17A to 17C are plan views of when the cap is slid downward in the blood testing device of a second embodiment, with FIG. 17A being a front view, FIG. 17B a side view (in FIG. 17B, the left side is the front face side, and the right side is the rear face side), and FIG. 17C a rear view;

FIGS. 18A to 18C are plan views of when the cap is slid upward in the blood testing device of a second embodiment, with FIG. 18A being a front view, FIG. 18B a side view (in FIG. 18B, the left side is the front face side, and the right side is the rear face side), and FIG. 18C a rear view;

FIGS. 19A and 19B are plan views of the main body support of the blood testing device, with FIG. 19A being a front view and FIG. 19B a side view (in FIG. 19B, the left side is the front face side, and the right side is the rear face side);

FIG. 20 is a plan view of the blood testing device as seen from the front face side;

FIG. 21 is a plan view of the main body as seen from the front face side;

FIG. 22 is a cross section of an adjustment hole and an adjustment protrusion, in which the cross sectional plane is perpendicular to the plane of view in FIG. 20 and parallel to the vertical direction in FIG. 20;

FIG. 23 is a plan view of the blood testing device as seen from the rear face side;

FIGS. 24A and 24B are plan views illustrating the adjustment of the distance between a support hole and a puncture hole with the blood testing device, and FIG. 24C is a detail plan view of the blood testing device as seen from the rear face side;

FIG. 25 is an oblique view of a conventional blood testing device;

FIG. 26 is an oblique view of the usage state of a conventional blood testing device; and

FIGS. 27A, 27B, 28A, 28B, 29A, 29B, 30A, 30B, 31A, 31B, 32A, 32B, 33A, 33B, 33C, 34A, 34B, 35A and 35B are diagrams of various aspects of the main body support.

### DETAILED DESCRIPTION OF AN EXAMPLE EMBODIMENT

Selected embodiments of the present invention will now be explained with reference to the drawings. It will be apparent to those skilled in the art from this disclosure that the following descriptions of the embodiments of the present invention are provided for illustration only and not for the purpose of limiting the invention as defined by the appended claims and their equivalents.

### (1) First Embodiment

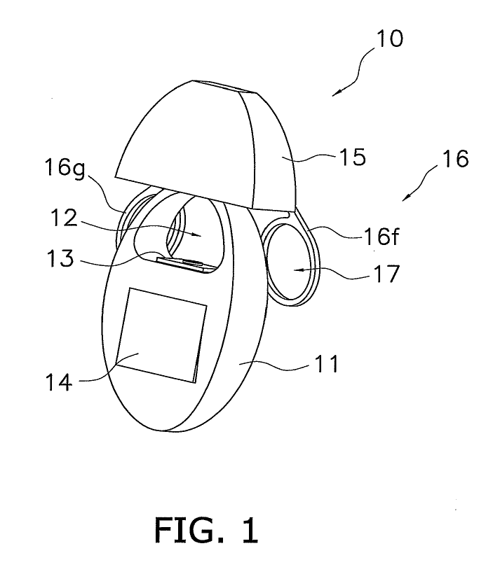

As shown in FIG. 1, a blood testing device 10 includes a main body 11, a display component 14, a cap 15, and main body supports 16 (16f and 16g).

### 1-1. Main Body 11

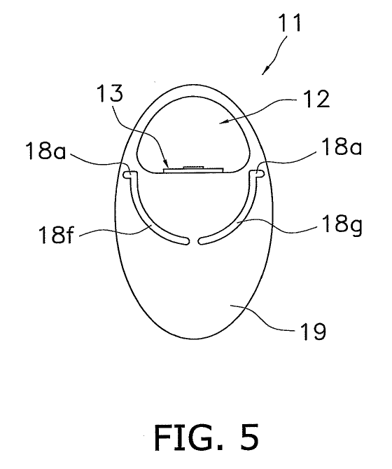

As shown in FIG. 5, the main body 11 has a casing 19 and a finger contact component 13.

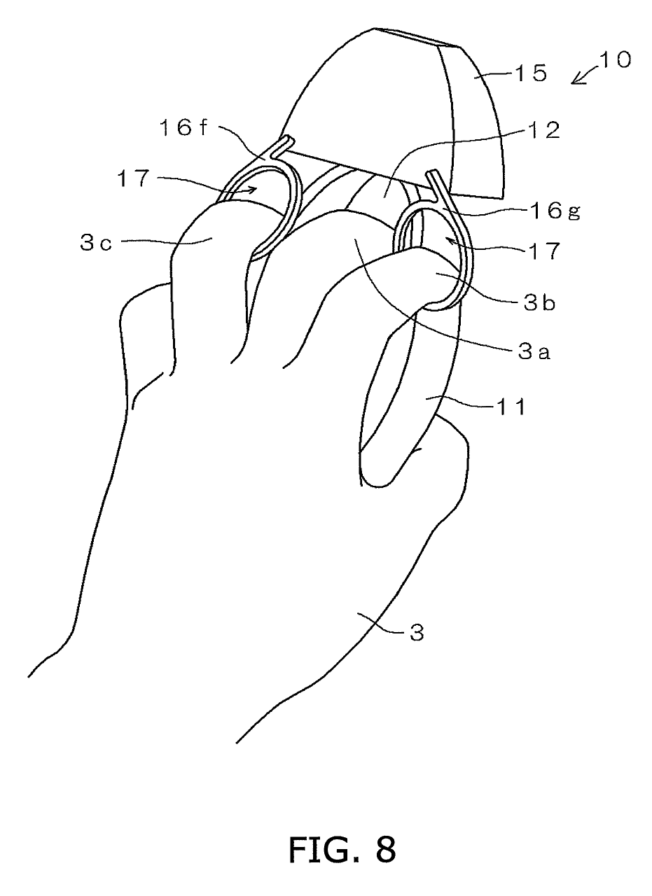

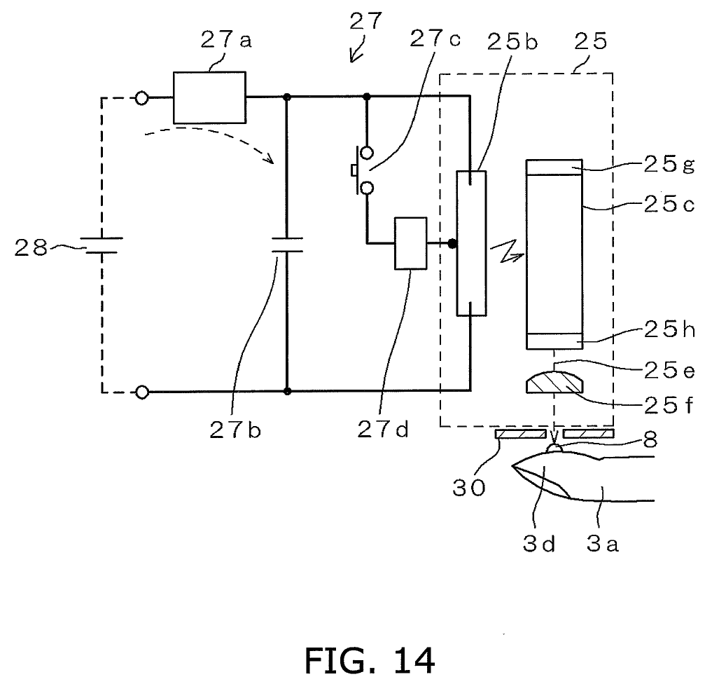

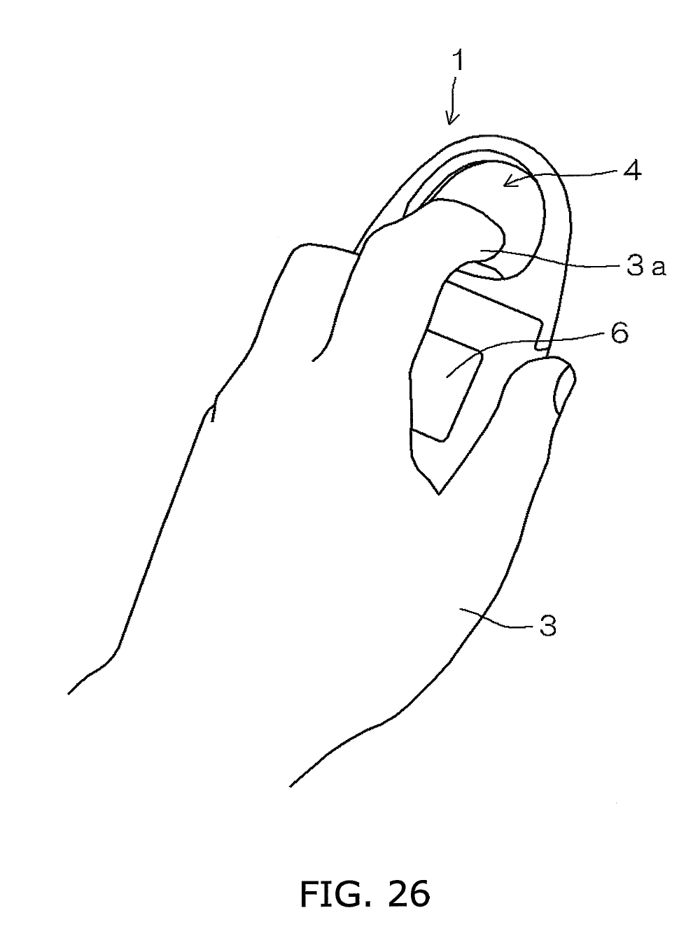

The casing 19 is the outer shell of the main body 11. The casing 19 has a flat, elliptical shape, and is the right size to fit in one hand. A puncture hole 12 is provided to a first end (the upper end in FIG. 1) of the casing 19 in the major axis direction of its elliptical shape. A finger 3a (see FIGS. 8 and 26) that will provide blood 8 (see FIG. 14) is inserted into the puncture hole 12.

Two grooves 18f and 18g are formed on the outer face of the casing 19, on the face (rear face) on the opposite side from the face where the display component 14 is provided. As shown in FIG. 5, the grooves 18f and 18g are depressions in the form of quarter circles. The second groove 18g is symmetrical with the first groove 18f with respect to the center vertical line of the main body 11 (a straight line extending up and down in FIG. 5). As shown in FIG. 5, the first groove 18f is one of two quarter-circle grooves obtained by dividing a semicircular groove in two at the center vertical line of the main body 11, while the second groove 18g is the other of these. The first groove 18f continues from the approximate center of the casing 19 up to the lower-left corner of the puncture hole 12 in FIG. 5. The second groove 18g continues from the approximate center of the casing 19 up to the lower-right corner of the puncture hole 12 in FIG. 5.

Horizontal parts 18a are provided at the upper ends of the grooves 18f and 18g (of the two ends of the groove 18f, the end near the puncture hole 12). As shown in FIG. 5, one horizontal part 18a is a depression that is contiguous with the first groove 18f, and extends horizontally to the left from the upper end of the first groove 18f. The other horizontal part 18a is a depression that is contiguous with the second groove 18g, and extends horizontally to the right from the upper end of the second groove 18g.

### 1-2. Finger Contact Component 13

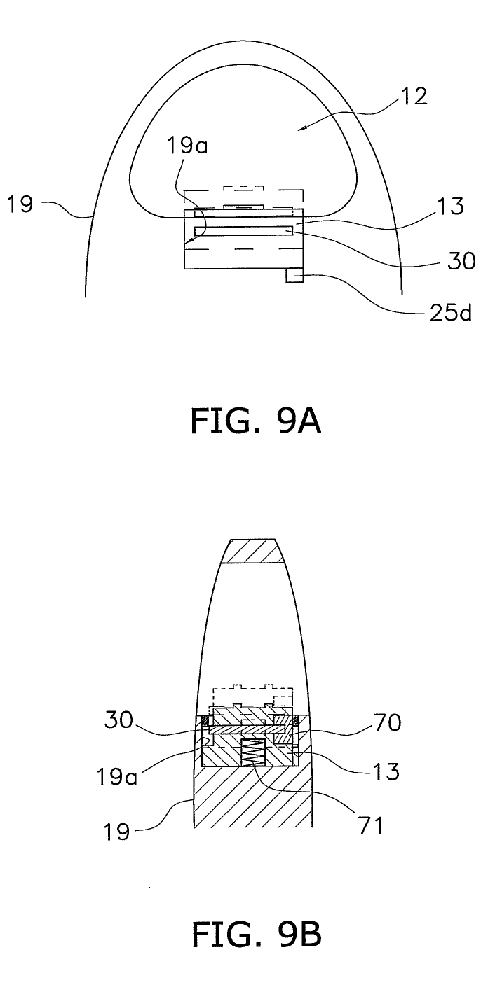

As shown in FIG. 9B, the finger contact component 13 is disposed in a hole 19a provided to the wall face at the lower part of the puncture hole 12. The finger contact component 13 is disposed so that part of it sticks out from the hole 19a. Also, the finger contact component 13 is provided so that it is able to move up and down. Inside the casing 19, an elastic member 71 is provided below the finger contact component 13. The elastic member 71 imparts an upward force to the finger contact component 13, and as a result, as shown in FIGS. 9A and 9B, when the finger contact component 13 is pressed down by the finger 3a of the user, it is able to return to its original position (dotted line).

Inside the casing 19, a puncture switch 25d is provided at a location that is below the finger contact component 13 and does not interfere with the elastic member 71. When the finger contact component 13 is pressed down, the puncture switch 25d is switched on. When the pressing of the finger contact component 13 is released, and the finger contact component 13 returns to its original position, the puncture switch 25d is switched off.

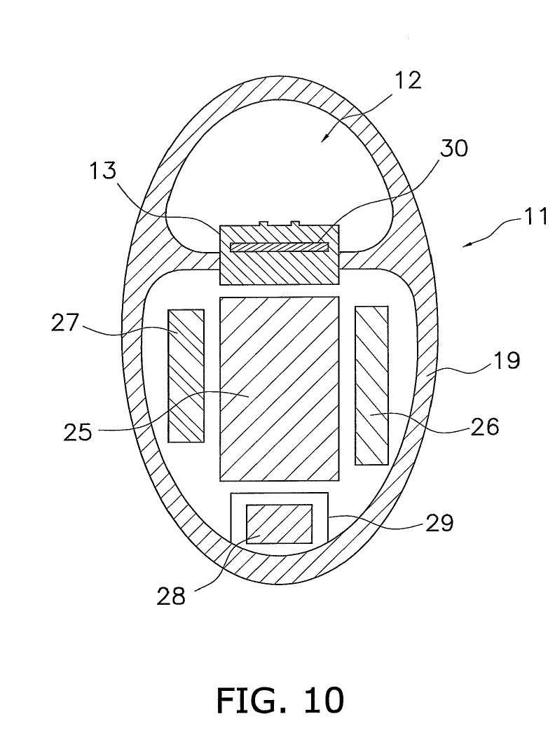

The finger contact component 13 has a structure that allows a blood sensor 30 to be mounted and removed (sensor receptacle 70) (see FIGS. 9B and 10).

### 1-3. Display Component 14

The display component 14 is provided to the front face of the main body 11. The display component 14 is a liquid crystal display panel.

### 1-4. Cap 15

The cap 15 is mounted at the upper end of the main body 11, so that it can slide up and down. The sliding of the cap 15 will be discussed below.

### 1-5. Main Body Supports 16

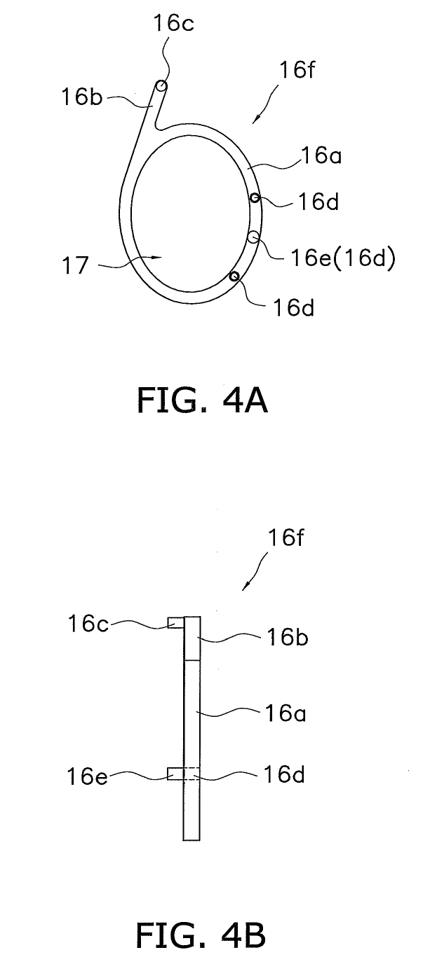

The blood testing device 10 is equipped with two main body supports (a first main body support 16f and a second main body support 16g). As shown in FIGS. 4A and 4B, the main body supports 16f and 16g each have an elliptical member 16a and a support component 16b formed on the elliptical member 16a. Of the main body supports 16, the shape of the second main body support 16g is in line symmetry with the shape of the first main body support 16f, so in FIGS. 4A and 4B, only the first main body support 16f is depicted, while the second main body support 16g is not.

As shown in FIG. 4A, the elliptical member 16a is a ring-shaped member; that is, a support hole 17 is provided to the elliptical member 16a.

As shown in FIGS. 4A and 4B, the support component 16b sticks out from the elliptical member 16a along a plane that includes the elliptical shape of the elliptical member 16a. A latching prong 16c is provided to the end of the support component 16b. The latching prong 16c sticks out from the support component 16b in a direction perpendicular to the plane that includes the elliptical member 16a.

As shown in FIGS. 4A and 4B, three screw holes 16d are formed in the elliptical member 16a. These screw holes 16d are equidistantly spaced. The number of screw holes 16d is not limited to three, but there are preferably two or more of the screw holes 16d.

A convex member 16e is installed in one of the screw holes 16d. In FIGS. 4A and 4B, of the three screw holes 16d, the convex member 16e is installed in the middle screw hole 16d. In a state in which the convex member 16e has been installed in the screw hole 16d, it sticks out in the same direction as the latching prong 16c, that is, toward the main body 11 side.

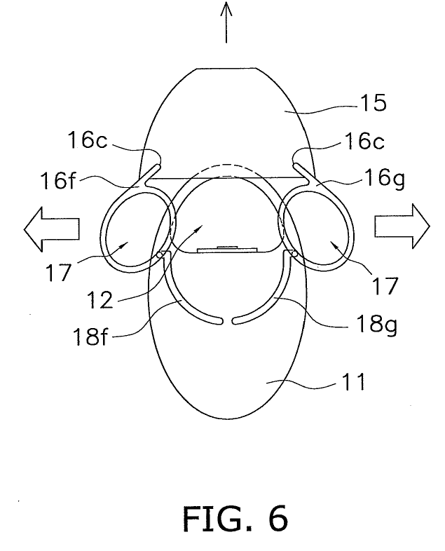

The specific disposition of the main body supports 16f and 16g is as follows. As shown in FIG. 6, the first main body support 16f is shaped like the numeral 6. When the blood testing device 10 is viewed from the rear, the first main body support 16f is mounted on the left side of the puncture hole 12. Depressions (not shown) are provided on the lower left and lower right of the cap 15. The latching prong 16c of the first main body support 16f is put in the lower-left depression of the cap 15. Thus the first main body support 16f is rotatably mounted in the lower-left depression of the cap 15 via the latching prong 16c. The convex member 16e of the first main body support 16f mates with the first groove 18f.

The shape of the second main body support 16g is symmetrical with the shape of the first main body support 16f with respect to the center vertical line of the main body 11 (a straight line extending up and down in FIG. 6). As shown in FIG. 6, the second main body support 16g is mounted on the right side of the puncture hole 12 when the blood testing device 10 is viewed from the rear. The latching prong 16c of the second main body support 16g fits into the depression at the lower right of the cap 15. The second main body support 16g is thus rotatably mounted in the lower-right depression of the cap 15 via the latching prong 16c. The convex member 16e of the second main body support 16g mates with the second groove 18g.

The main body supports 16f and 16g are provided so that they rotate in conjunction with the sliding of the cap 15. The movement of the main body supports 16f and 16g will be described below.

### 1-6. Sliding of Cap

As discussed above, the cap 15 is provided so that it slides up and down. Thus, the user can slide the cap 15 up when a finger is to be punctured, and slide the cap 15 down otherwise.

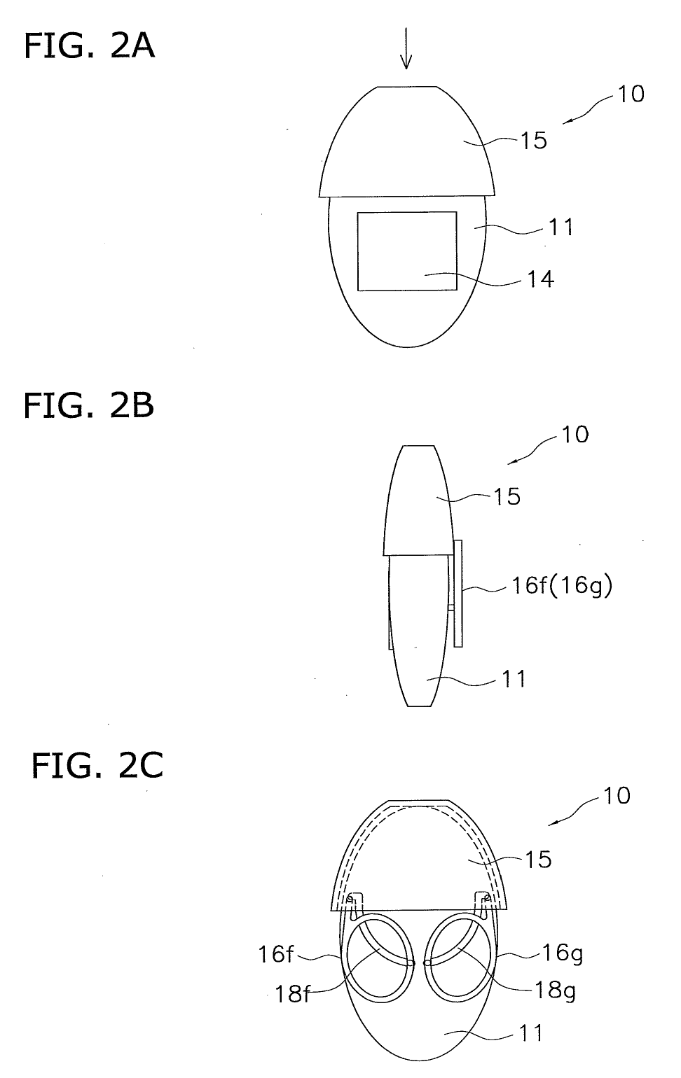

In FIGS. 2A to 2C, the cap 15 has been slid down. That is, in these drawings, the cap 15 is in its closed state.

In FIG. 2A, the puncture hole 12 is blocked by the cap 15. This prevents a finger from accidentally being inserted into the puncture hole 12. That is, a finger cannot be punctured except when it is supposed to be. The blood testing device 10 thus has excellent safety. Even when the cap 15 is closed, the display component 14 is not covered by the cap 15. Thus, even if the user closes the cap 15, a blood sugar value that has already been measured can still be viewed.

As shown in FIG. 2B, the main body supports 16f and 16g are provided on the outside of the cap 15. Thus, the blood testing device 10 in this aspect affords a smaller cap 15.

As shown in FIG. 2C, in a state in which the cap 15 has been slid down, the main body supports 16f and 16g are stowed on the rear face of the main body 11, and do not stick out to the left and right of the main body 11. Thus, the device is easy to carry around in a state in which the cap 15 has been slid down.

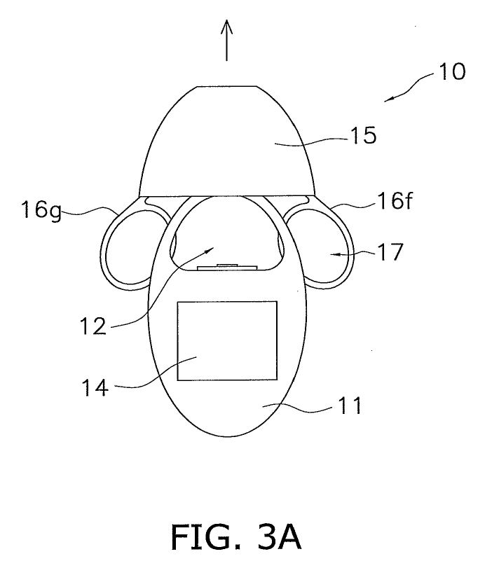

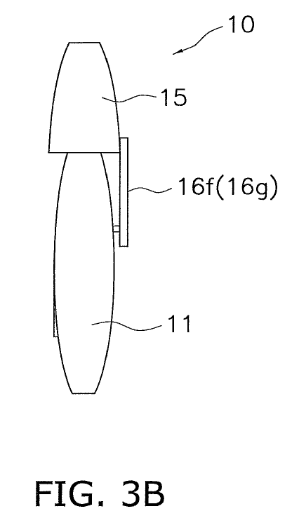

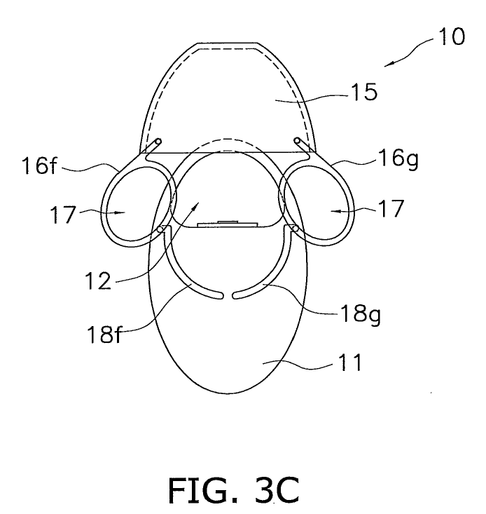

The main body supports 16f and 16g change from the state in FIGS. 3A to 3C (in which the cap 15 has been slid up) to the position in FIGS. 2A to 2C by sliding in the arc-shaped grooves 18 formed on the rear face of the main body 11.

As shown in FIG. 3A, in a state in which the cap 15 has been slid up, the puncture hole 12 is exposed from the cap 15. That is, the puncture hole 12 is open. Therefore, the user can insert the finger 3a into the puncture hole 12 for puncture.

As shown in FIG. 3B, the main body supports 16f and 16g are provided outside the main body 11 and the cap 15. The main body supports 16f and 16g define the spacing between the main body 11 and the cap 15.

As shown in FIG. 3C, the main body supports 16f and 16g open (stick out) to the left and right of the puncture hole 12. That is, the support holes 17 are disposed to the outside on the left and right of the main body 11, and the support holes 17 are not blocked by the main body 11. Therefore, when the user wants to use the device, he can insert fingers 3b and 3c (those that are not to be punctured) into the support holes 17.

The main body supports 16f and 16g change from the state in FIGS. 2A to 2C (in which the cap 15 has been slid down) to the position in FIGS. 3A to 3C by sliding in the arc-shaped grooves 18 formed on the rear face of the main body 11.

As shown in FIG. 6, when the cap 15 is slid up, the convex members 16e slide in the grooves 18, and the main body supports 16f and 16g are moved to the outside (to the left and right). When the main body supports 16f and 16g move to the outside of the main body 11, the support holes 17 move from the rear face of the main body 11 to the left and right sides of the puncture hole 12 in FIG. 6.

The convex members 16e move the horizontal parts 18a outward (laterally) upon moving to the upper ends of the grooves 18. Thus, when the convex members 16e are disposed in the horizontal parts 18a, they are prevented from moving below the cap 15 under their own weight.

The latching prongs 16c are biased outward and rotatably mounted at the lower part of the cap 15. Therefore, in a state in which the convex members 16e are fitted into the horizontal parts 18a, the main body supports 16f and 16g are biased outward, which prevents them from moving below the cap 15 under their own weight.

The spacing between the puncture hole 12 and the support holes 17 can be varied according to the position of the convex members 16e.

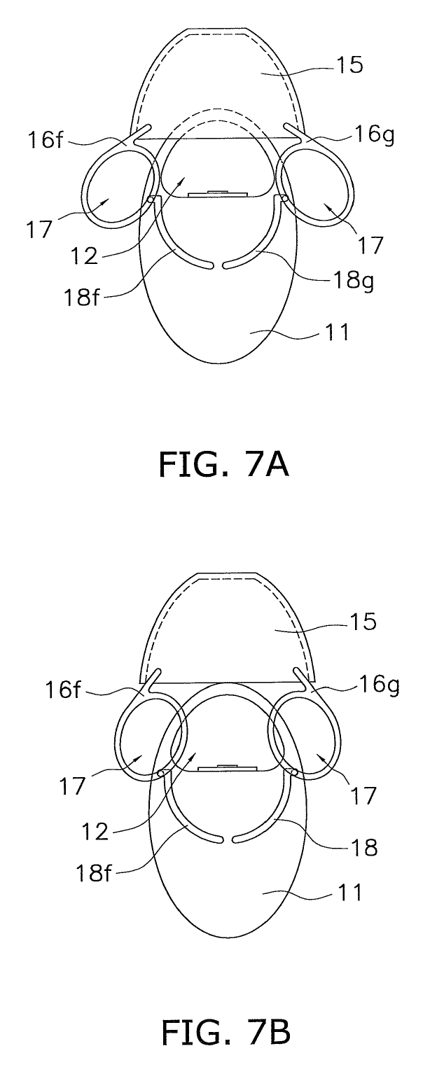

For example, as shown in FIG. 7A, if the convex members 16e (see FIG. 4A) are installed in the uppermost of the three screw holes 16d, the spacing between the puncture hole 12 and the support holes 17 is wider. As shown in FIG. 7B, if the convex members 16e (see FIG. 4A) are installed in the lowermost of the three screw holes 16d, the spacing between the puncture hole 12 and the support holes 17 is narrower.

In FIG. 8, the blood testing device 10 is held in the left hand 3. At this point, the finger 3a (the finger to be punctured; the middle finger in FIG. 8) is inserted into the puncture hole 12. One of the fingers not to be punctured, the finger 3b (the index finger in FIG. 8), is inserted into the support hole 17 formed in the second main body support 16g. Another of the fingers not to be punctured, the finger 3c (the ring finger in FIG. 8), is inserted into the support hole 17 formed in the first main body support 16f.

As discussed above, the spacing between the puncture hole 12 and the support holes 17 can be adjusted. That is, the spacing between the puncture hole 12 and the support holes 17 is adjusted according to the size of the user's hand 3. Also, since the fingers 3b and 3c (fingers that are not to be punctured) adjacent to the finger 3a (to be punctured) are inserted into the support hole 17 of the first main body support 16f and the support hole 17 of the second main body support 16g, respectively, the finger contact component 13 is stably attached to the finger 3a.

Thus, the position of the blood testing device 10 with respect to the hand 3 is stably fixed. Therefore, the blood 8 that seeps out after puncture is reliably provided to the blood sensor 30. As a result, accurate blood testing is accomplished. Also, when the non-puncture fingers 3b and 3c are inserted in the support holes 17 provided adjacent to the puncture hole 12, the main body 11 is stably held by the hand 3. This keeps the blood testing device 10 from being dropped from the hand 3.

### 1-7. Internal Structure of Main Body 11

As shown in FIG. 10, in addition to the constitution discussed above, the main body 11 has a laser unit 25, a measurement circuit 26, and a high voltage generating circuit 27. A battery compartment 29 that holds a battery 28 for supplying power to the constituent elements is provided to the lower part of the main body 11. As discussed above, the blood sensor 30 can be installed in and removed from the finger contact component 13.

### Blood Sensor 30

The blood sensor 30 is a member that can be installed in and removed from the blood testing device 10. Thus, the blood sensor 30 may be considered not to be included in the blood testing device 10, or may be considered to be part of the blood testing device 10.

As shown in FIG. 9B, the sensor receptacle 70 is provided to the finger contact component 13. The blood sensor 30 is removably mounted in the sensor receptacle 70.

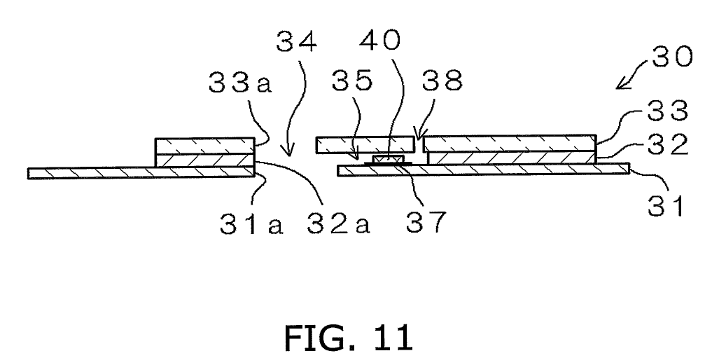

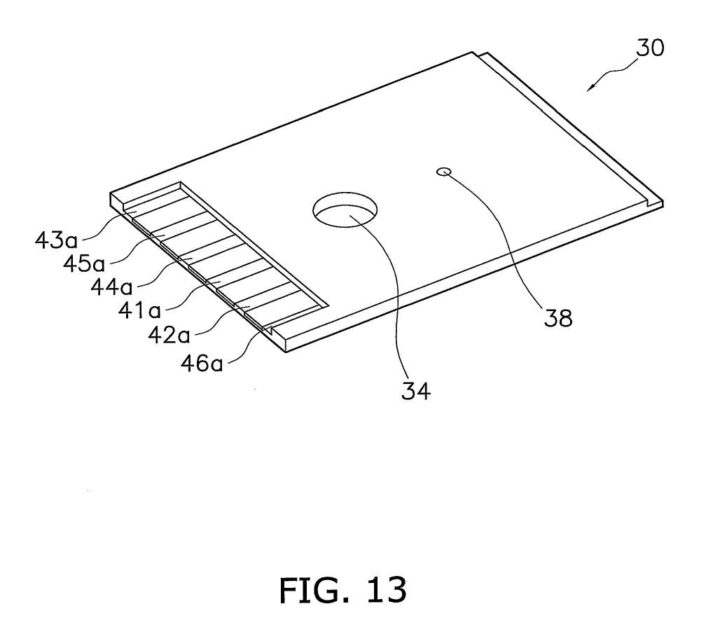

As shown in FIGS. 11 and 13, the blood sensor 30 has a substrate 31, a spacer 32 that is affixed to the upper face of the substrate 31 (the side closer to the finger 3a), and a cover 33 affixed to the upper face of the spacer 32. The blood sensor 30 also includes a reservoir 34, a supply path 35, a detector 37, an air hole 38, a reagent 40, detecting electrodes 41 to 45, an identifier 46, and connecting electrodes 41a to 46a.

As shown in FIG. 11, the reservoir 34 is a through-hole that passes through the substrate 31, the spacer 32, and the cover 33. In other words, the through-hole 34 is formed by a substrate hole 31a formed in the approximate center of the substrate 31, a spacer hole 32a formed in the spacer 32, and a cover hole 33a formed in the cover 33. The substrate hole 31a and the spacer hole 32a communicate, and the spacer hole 32a and the cover hole 33a communicate. The finger 3a is placed in the opening of the reservoir 34 in the cover 33. The reservoir 34 holds the blood 8 that seeps out after puncture.

The supply path 35 is a gap (capillary) provided by the spacer 32 between the substrate 31 and the cover 33. The first end of the supply path 35 leads to the reservoir 34. The supply path 35 guides the blood 8 held in the reservoir 34 to the detector 37 by capillary action. The second end of this supply path 35 is linked to the air hole 38. In this aspect, the volume of the reservoir 34 is 0.904 μL, and the volume of the supply path 35 is 0.144 μL. However, the volumes here are not limited to these values, as long as enough blood for the test is guided from the reservoir 34 to the detector 37.

The air hole 38 is a hole that goes through the cover 33 and the spacer 32. That is, the air hole 38 leads from the upper face of the cover 33 to the supply path 35.

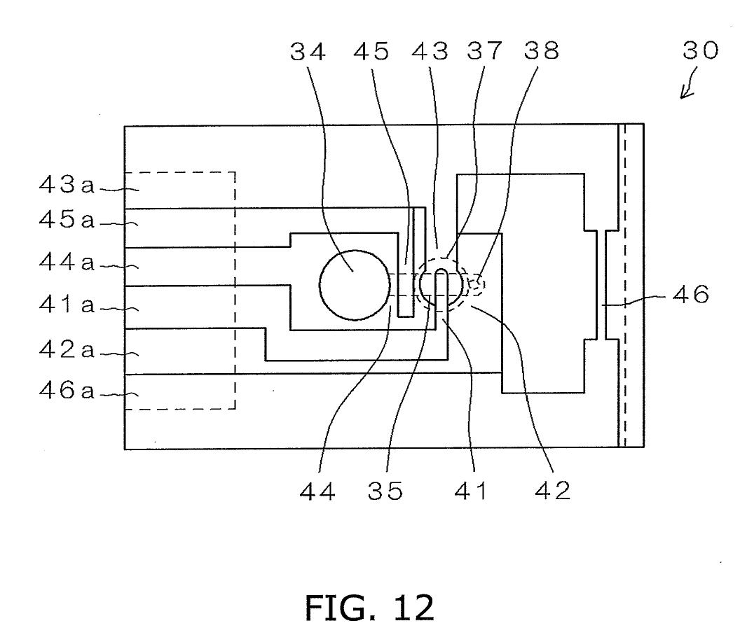

As shown in FIG. 12, the detector 37 has detecting electrodes 41 and 42.

The reagent 40 is placed on the detector 37. This reagent 40 is formed by adding and dissolving PQQ-GDH (0.1 to 5.0 U/sensor), potassium ferricyanide (10 to 200 mM), maltitol (1 to 50 mM), and taurine (20 to 200 mM) in a 0.01 to 2.0 wt % CMC aqueous solution to prepare a reagent aqueous solution, putting a drop of this on detecting electrodes 41 and 43 (see FIG. 12) formed on the substrate 31, and drying.

The detecting electrodes 41 to 45 and connecting electrodes 41a to 45a are formed on the upper surface of the substrate 31. The detecting electrode 41 and the connecting electrode 41a, the detecting electrode 42 and the connecting electrode 42a, the detecting electrode 43 and the connecting electrode 43a, the detecting electrode 44 and the connecting electrode 44a, and the detecting electrode 45 and the connecting electrode 45a are each formed by continuous conductive layers. In other words, the connecting electrodes 41a to 45a are connected to the respective detecting electrodes 41 to 45.

The connecting electrode 46a and the identifier 46 are formed on the upper face of the substrate 31. As shown in FIGS. 12 and 13, the connecting electrodes 41a to 46a are disposed so as to line up at one end of the blood sensor 30. The identifier 46 is a conductor formed between the connecting electrode 43a and the connecting electrode 46a.

The detecting electrodes 41 to 45, the identifier 46, and the connecting electrodes 41a to 46a can be formed by laser working a conductive layer formed by sputtering or vapor deposition, using a material such as gold, platinum, or palladium.

The layout of the electrodes is as follows. The detecting electrode 44 connected to the connecting electrode 44a, the detecting electrode 45 connected to the connecting electrode 45a, the detecting electrode 43 reconnected to the connecting electrode 43a, the detecting electrode 41 connected to the connecting electrode 41a, and the detecting electrode 42 connected to the connecting electrode 42a are provided in the lengthwise direction along the supply path 35 in that order, going from the reservoir 34 toward the air hole 38. The reagent 40 (see FIG. 11) is placed on the detecting electrodes 41 and 43. The identifier 46 is disposed at a location that is farther from the reservoir 34 than the detecting electrode 42. The identifier 46 is disposed at a location that is away from the supply path 35.

A controller 26g (discussed below) can determine whether or not the blood sensor 30 has been mounted to the sensor receptacle 70 from whether or not there is electrical conduction between the connecting electrode 43a and the connecting electrode 46a. Specifically, when this blood sensor 30 is placed in the sensor receptacle 70, if electrical conduction is detected between the connecting electrode 43a and the connecting electrode 46a, it is determined that the blood sensor 30 has been properly mounted on sensor receptacle 70.

It is also possible to store information about a calibration curve that is used, or to store manufacturing information, by varying the electrical resistance of the identifier 46 with the controller 26g. Therefore, this information can be used to conduct a more precise blood test.

Since the blood sensor 30 is constituted as above, the blood 8 adhering to the reservoir 34 is guided by capillary action through the supply path 35, passing through the detecting electrodes 45 and 43 in that order, to the top of the detecting electrode 42. Once the blood 8 has reached the detecting electrode 42, enough blood 8 for measurement reaches the detecting electrodes 41 and 43. This is because the detecting electrodes 41 and 43 are disposed closer to the reservoir 34 than the detecting electrode 42. The blood 8 reacts with the reagent 40. After this reaction, a specific voltage is applied to the electrodes 41 and 43, which generates a tiny current. This current flows to the connecting electrodes 41a and 43a.

1-7-a. Laser Unit 25

The laser unit 25 is an example of a puncture component. The laser unit 25 is disposed at a location opposite the finger contact component 13 inside the casing 19.

As shown in FIG. 14, the laser unit 25 has a lamp 25b, a laser rod 25c, and a lens 25f. The laser rod 25c is provided near the lamp 25b. The laser rod 25c is made of Er:YAG (yttrium-aluminum-garnet). The lens 25f is disposed in front of the laser rod 25c (on the side closer to the blood sensor 30 than the laser rod 25c).

A total reflection film 25g is formed at the end face of the laser rod 25c that is on the opposite side from the end face on the laser emission side. A partial transmission film 25h is formed at the end face of the laser rod 25c on the laser emission side.

A total reflection mirror may be disposed in place of the total reflection film 25g, and a partial transmission mirror instead of the partial transmission film 25h.

The reflectivity of a total reflection mirror is at least 99.5%. A total reflection mirror is, for example, a mirror coating of aluminum with a protective film, or a coating of a low-absorption, dielectric, multilayer film. Meanwhile, the reflectivity of a partial reflection mirror is 80 to 95%. A partial reflection mirror is, for example, a coating of a low-absorption, dielectric, multilayer film. Either type of mirror may be a multilayer film in which layers of SiO₂ are alternated with ZrO₂.

The blood testing device 10 may also be equipped with a puncture component that punctures the finger 3a with a needle, instead of the laser unit 25.

1-7-b. Measurement Circuit 26

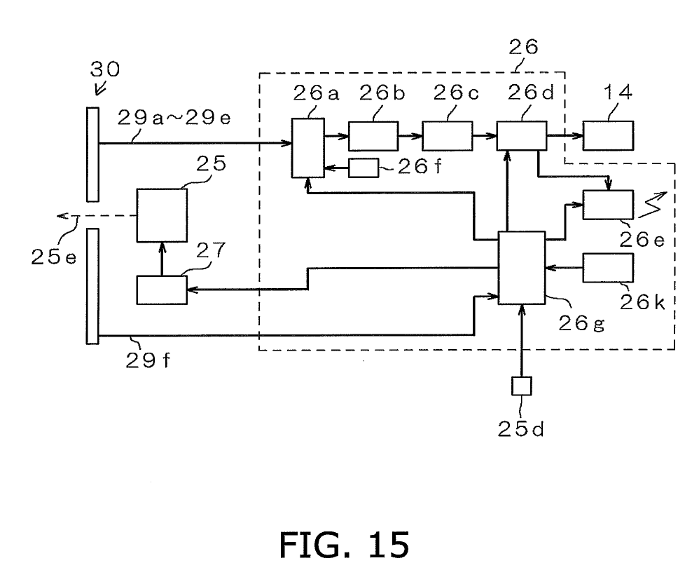

As shown in FIG. 15, the measurement circuit 26 includes a switching circuit 26a, a current/voltage converter 26b, an analog/digital converter (hereinafter referred to as an A/D converter) 26c, a computer 26d, a transmitter 26e, a reference voltage supply 26f, the controller 26g, and a timer 26k. The computer 26d and the controller 26g are constituted by a CPU (central processing unit) and a recording medium. The functions of the computer 26d and the controller 26g are carried out when the CPU reads and executes programs stored in the recording medium.

The switching circuit 26a is connected to the connecting electrodes 41a to 45a (FIG. 12) of the blood sensor 30 via contact members 29a to 29e provided to the sensor receptacle 70. The output terminal of the switching circuit 26a is connected to the input terminal of the current/voltage converter 26b. The output terminal of the current/voltage converter 26b is connected to the input terminal of the A/D converter 26c. The output terminal of the A/D converter 26c is connected to the input terminal of the computer 26d. The output terminal of the computer 26d is connected to the display component 14 and the transmitter 26e. Also, the reference voltage supply 26f is connected to the switching circuit 26a.

The output terminal of the controller 26g is connected to the high voltage generating circuit 27, the control terminal of the switching circuit 26a, the computer 26d, and the transmitter 26e. The input terminal of the controller 26g is connected to the puncture switch 25d, the timer 26k, and the contact member 29f. The contact member 29f is connected to the connecting electrode 46a.

1-7-c. High Voltage Generating Circuit 27

The high voltage generating circuit 27 supplies high voltage to the laser unit 25.

As shown in FIG. 14, the high voltage generating circuit 27 has a booster circuit 27a, a capacitor 27b, a trigger switch 27c, and a trigger circuit 27d.

The booster circuit 27a is connected to the battery 28. The capacitor 27b is connected to the output terminal of the booster circuit 27a. The trigger switch 27c is connected to the capacitor 27b. The trigger circuit 27d is connected to the output terminal of the trigger switch 27c.

The two terminals of the capacitor 27b are connected to the two electrodes (pair of main electrodes) of the lamp 25b of the laser unit 25. The output terminal of the trigger circuit 27d is connected to the trigger electrode of the lamp 25b. The electrostatic capacity of the capacitor 27b is 200 to 450 μF, and the voltage resistance is 200 to 400 V. The trigger switch 27c is an IGBT (insulated gate bipolar transistor). This trigger switch 27c is switched on and off by the output of the puncture switch 25d shown in FIG. 9.

### 1-8. Operation of Blood Testing Device

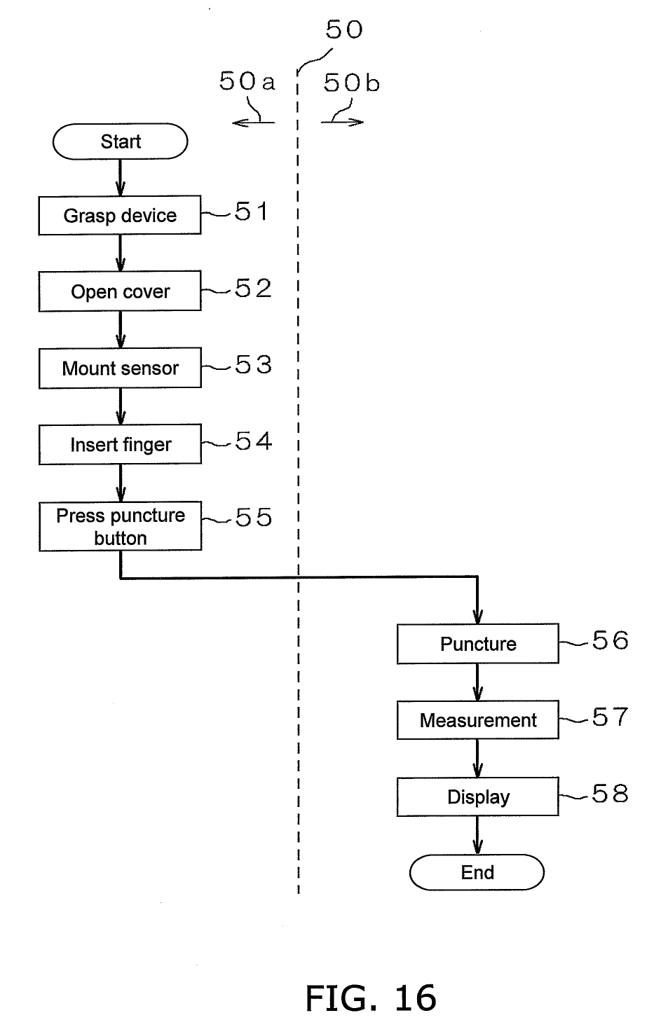

In FIG. 16, the steps on the left side 50a of the dotted line 50 are user operations, while the steps on the right side 50b are operations of the blood testing device 10.

1-8-a. Steps 51 to 55

As shown in FIG. 16, the user holds the blood testing device 10 (step 51). Then, the user slides the cap 15 upward (step 52). When the cap 15 slides upward, the finger contact component 13 appears. The user mounts a new blood sensor 30 to the finger contact component 13 (step 53).

Then, as shown in FIG. 8, the user inserts the finger 3a (the finger to be punctured) into the puncture hole 12, and inserts the fingers 3b and 3c (fingers not to be punctured) into the support holes 17 (step 54). It does not matter whether the finger 3a is inserted first, or the fingers 3b and 3c.

The user then presses the finger contact component 13 with the puncture finger 3a (step 55). Pressing the finger contact component 13 switches on the puncture switch 25d.

1-8-b. Steps 56 to 58

Before step 56, the supply of voltage from the battery 28 is commenced at the point when the puncture switch 25d of the blood testing device 10 is switched on. The voltage supplied from the battery 28 is boosted by the booster circuit 27a, and charges the capacitor 27b. The operation of the booster circuit 27a is controlled by the controller 26g, and this controls the charging voltage. The depth of puncture is controlled by the level of the charging voltage.

The voltage charged to the capacitor 27b is supplied to the electrodes of the lamp 25b. When the finger contact component 13 is pressed with the finger 3a (when the puncture switch 25d is pressed) in a state in which this voltage has been boosted to a predetermined level, 0.5 second later light is emitted from the lamp 25b. This time is measured by the timer 26k. The emission of light from the lamp 25b causes the laser beam 25e to be emitted from the laser rod 25c. The laser beam 25e passes through the lens 25f and the blood sensor 30 and punctures the skin 3d of the puncture finger 3a (step 56). The blood 8 seeps out of the skin 3d.

When the skin 3d is punctured, the blood 8 seeps out. This blood 8 is captured in the blood sensor 30. The blood sensor 30 generates a current corresponding to the glucose concentration in the blood, and sends this current as an electrical signal (measurement signal) to the measurement circuit 26.

More specifically, at a command from the controller 26g, the switching circuit 26a connects the detecting electrode 41 to the current/voltage converter 26b. Also, the switching circuit 26a connects the detecting electrode 42 to the reference voltage supply 26f. The detecting electrode 42 detects the inflow of the blood 8. A specific voltage is applied between the detecting electrodes 41 and 42.

In this state, when the blood 8 flows in, current flows between the detecting electrodes 41 and 42. This current is converted into voltage by the current/voltage converter 26b, and the voltage value thereof is converted into a digital value by the A/D converter 26c. This value is outputted to the computer 26d. The computer 26d determines that enough of the blood 8 has flowed in once this digital value exceeds a specific value.

Next, the concentration of glucose (blood sugar value), which is a blood component, is measured. First, at a command from the controller 26g, the switching circuit 26a connects the detecting electrode 41 to the current/voltage converter 26b. The switching circuit 26a connects the detecting electrode 43 to the reference voltage supply 26f. A specific voltage is applied between the detecting electrodes 41 and 43. In this blood sugar value measurement, the detecting electrode 41 functions as a working electrode, and the detecting electrode 43 functions as a counter electrode.

The current flowing between the detecting electrodes 41 and 43 is converted into voltage by the current/voltage converter 26b. This voltage value is converted into a digital value by the A/D converter 26c. This digital value is outputted to the computer 26d. The computer 26d converts this digital value into a glucose concentration.

The blood sugar value is thus calculated by the measurement circuit 26 (step 57). The calculated result is displayed on the display component 14 in step 58.

The measurement of the blood sugar value was used as an example above, but if the component of the reagent 40 is changed, the blood testing device 10 can also be applied to the measurement of lactic acid or cholesterol in the blood, instead of measuring glucose.

### (2) Second Embodiment

Of the members shown in the drawings referred to below, those that have the same function as in the first embodiment will be numbered the same and may not be described again.

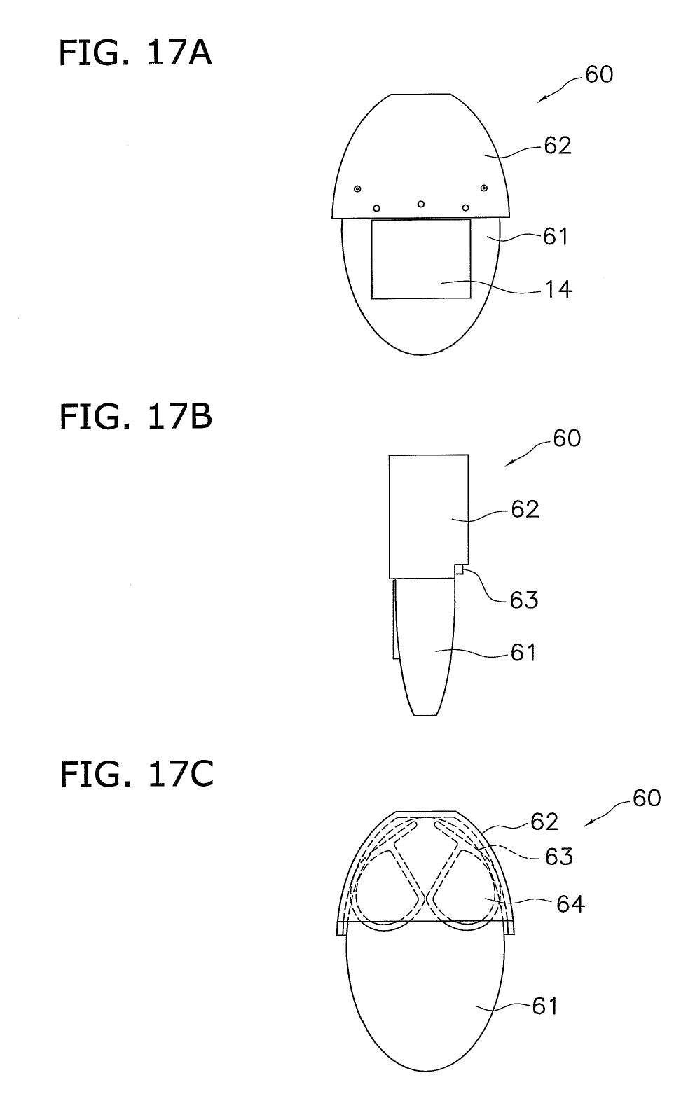

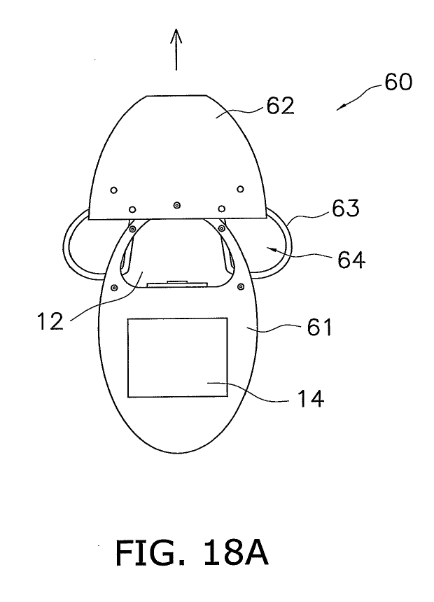

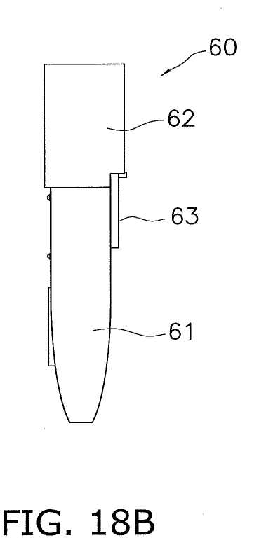

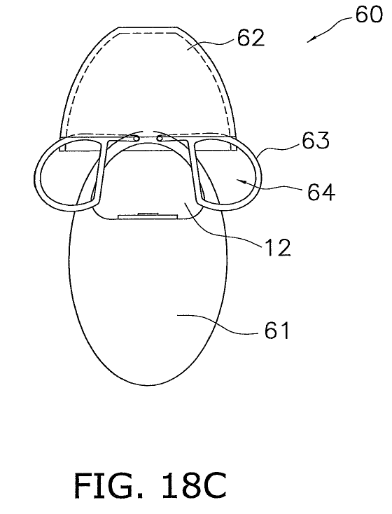

As shown in FIGS. 17A to 17C and FIGS. 18A to 18C, the blood testing device 60 in this embodiment is equipped with a main body 61 instead of the main body 11, with a cap 62 instead of the cap 15, and with main body supports 63 (63f and 63g) instead of the main body support 16, but in all other respects the constitution is the same as that of the blood testing device 10 in the first embodiment.

With the blood testing device 60, the main body supports 63 are stowed between the main body 61 and the cap 62. That is, when not being used, the main body support 63 does not stick out from the cap 62. The user can put the blood testing device 60 in his pocket, etc., when carrying around the blood testing device 60. Since the blood testing device 60 does not snag on the pocket here, it is easier to carry.

In FIGS. 17A to 17C, the cap 62 has been slid down. In this state, the puncture hole 12 is blocked by the cap 62. Also, support holes 64 are stowed within the cap 62. The support holes 64 correspond to the support holes 17 in the first embodiment.

As shown in FIG. 17A, since the puncture hole 12 is blocked by the cap 62, this prevents the puncture finger 3a from accidentally being inserted into the puncture hole 12. Thus, the blood testing device 60 has excellent safety. On the other hand, the display component 14 is not covered by the cap 62. Thus, with the blood testing device 60 in this state, the user can still check a blood sugar value that has already been measured.

As shown in FIG. 17B, the main body supports 63 are provided between the main body 61 and the cap 62. As discussed above, when the blood testing device 60 is stowed in a pocket and carried around, the main body supports 63 will not snag on the pocket, making this device suited to portable applications.

As shown in FIG. 17C, the main body supports 63 are stowed between the cap 62 and the rear face of the main body 61. Therefore, the blood testing device 60 can be more compact.

In FIGS. 18A to 18C, the cap 62 has been slid up. In this state, the puncture hole 12 is not closed off by the cap 62, and is open instead. Therefore, the user can insert the finger 3a (the finger to be punctured) into the puncture hole 12 and measure the blood sugar value with the blood testing device 60. Also, the support holes 64 are disposed at locations adjacent to the puncture hole 12. The user can insert the fingers 3b and 3c (fingers not to be punctured) into these support holes 64. Therefore, the user can hold the blood testing device 60 stably. This prevents the blood testing device 60 from being dropped during measurement.

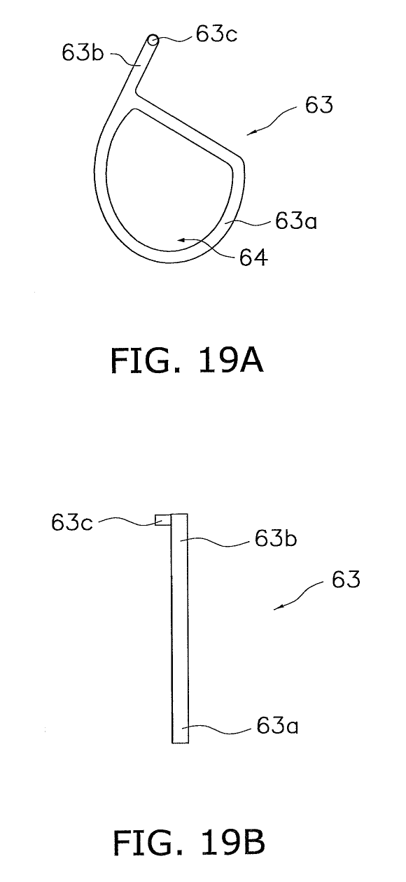

As shown in FIG. 19A, the main body supports 63 each have a semicircular (fan-shaped) member 63a and a support 63b formed above the semicircular member 63a. The semicircular member 63a has a support hole 64. A latching prong 63c is formed at the upper end of the support 63b. This latching prong 63c is latched to the upper part of the main body 61.

As shown in FIGS. 19A and 19B, the latching prong 63c is formed at the upper end of the support 63b.

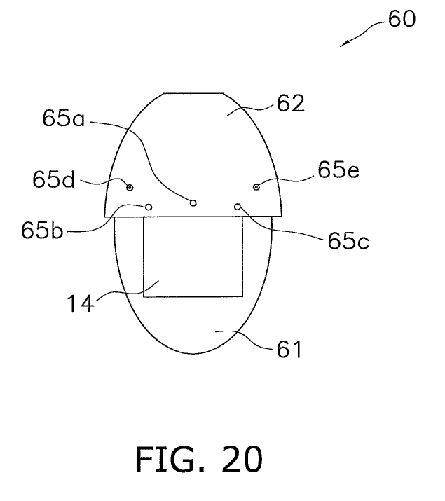

As shown in FIG. 20, when not in use, the upper part of the main body 61 is covered by the cap 62. A plurality of adjustment holes 65a to 65e (an example of a first latching component) are provided on the front face side of the cap 62.

The adjustment hole 65a is provided in the center of the cap 62 in the horizontal direction in FIG. 20, and at the lower portion of the cap 62. The adjustment holes 65b and 65d are provided on the left of the adjustment hole 65a in that order starting from the adjustment hole 65a. The adjustment hole 65c is disposed at a location that is symmetrical with the adjustment hole 65b, and the adjustment hole 65e at a location that is symmetrical with the adjustment hole 65d, with respect to the center vertical line of the cap 62 (a straight line extending up and down in FIG. 20 and passing through the adjustment hole 65a).

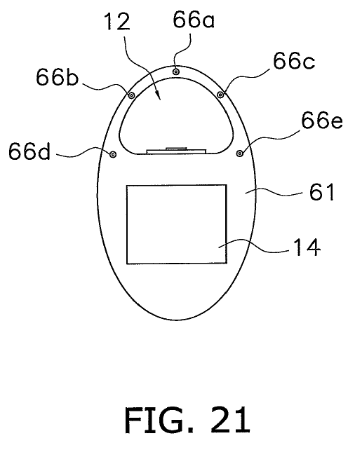

As shown in FIG. 21, the puncture hole 12 is provided at the upper part of the main body 61. Also, on the main body 61, a plurality of adjustment protrusions 66a to 66e (an example of a second latching component) are provided around the puncture hole 12.

The adjustment protrusion 66a is provided above the puncture hole 12, and is provided in the center of the main body 61 with respect to the horizontal direction in FIG. 21. The adjustment protrusions 66b and 66d are provided to the left of the adjustment protrusion 66a, in that order starting from the adjustment protrusion 66a. The adjustment protrusion 66c is disposed at a location that is symmetrical with the adjustment protrusion 66b, and the adjustment protrusion 66e at a location that is symmetrical with the adjustment protrusion 66d, with respect to the center vertical line of the main body 61 (a straight line extending up and down in FIG. 21 and passing through the adjustment protrusion 66a).

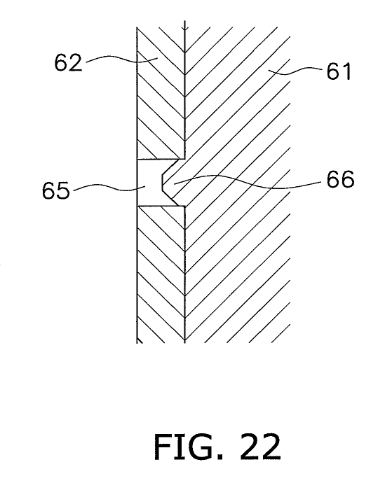

As shown in FIG. 22, the adjustment protrusions 66 (66a to 66e) mate with the adjustment holes 65 (65a to 65e) corresponding to the respective adjustment protrusions 66. Thus, the adjustment protrusions 66 are removably latched in the adjustment holes 65.

In a state in which the cap 62 is closed (FIG. 17A), the adjustment protrusion 66d is latched in the adjustment hole 65d, and the adjustment protrusion 66e is latched in the adjustment hole 65e. When the cap 62 is then slid upward, the adjustment protrusion 66b is latched in the adjustment hole 65b, and the adjustment protrusion 66c in the adjustment hole 65c. When the cap 62 is slid further upward, the adjustment protrusion 66a is latched in the adjustment hole 65a (FIG. 18A).

When the cap 62 is slid down, the latching state changes in the reverse order.

Also, the adjustment holes 65 may instead be provided to the main body 61, and the adjustment protrusions 66 to the cap 62.

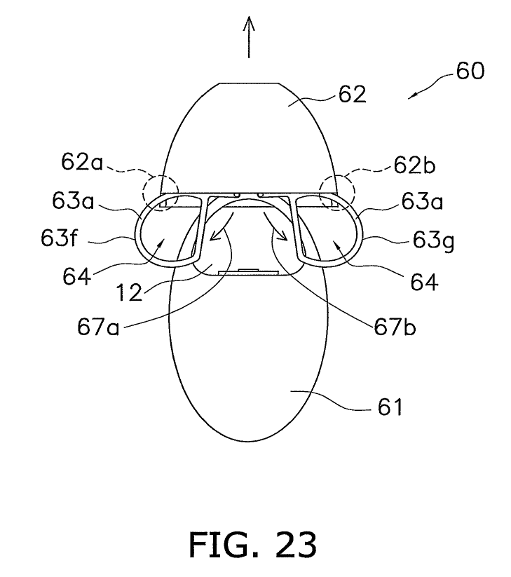

As shown in FIG. 23, when the cap 62 reaches its uppermost position, the puncture hole 12 appears. That is, the puncture hole 12 is open in FIG. 23.

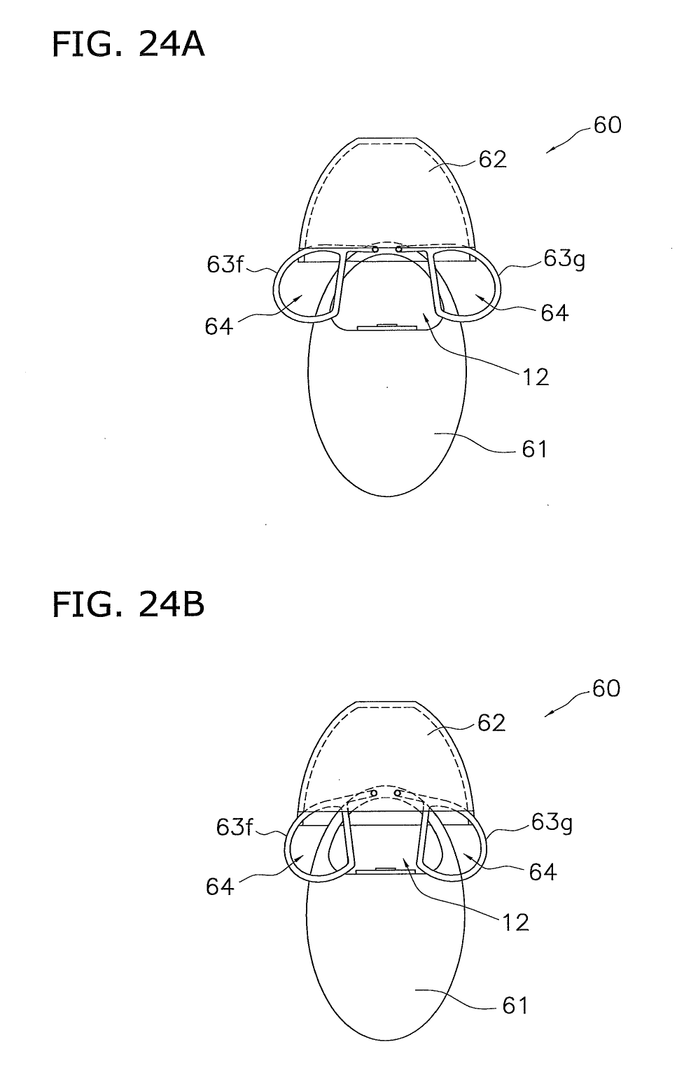

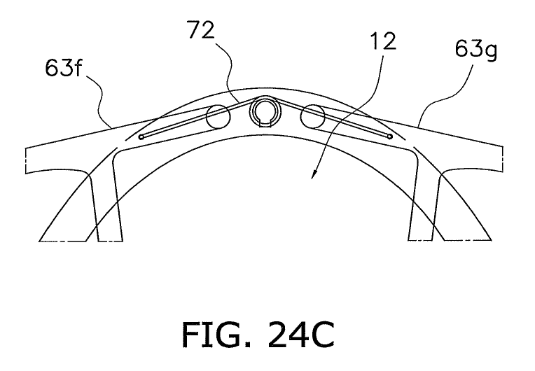

On the rear face, the two main body supports 63f and 63g are mounted at the upper part of the main body 61. As shown in FIG. 24C, the main body supports 63f and 63g are biased by a coil spring 72 in their respective outward directions (the directions of the arrows 67a and 67b). Therefore, the semicircular member 63a of the main body support 63f is always in contact with the lower left end 62a of the cap 62, and the semicircular member 63a of the main body support 63g is always in contact with the lower right end 62b.

Therefore, when the cap 62 is slid upward, the main body supports 63f and 63g slide outward while in contact with the ends 62a and 62b of the cap 62. Specifically, the spacing between the left and right support holes 64 varies according to the vertical position of the cap 62.

In FIG. 24A, the adjustment protrusion 66a of the main body 61 (see FIG. 21) is latched to the adjustment hole 65a of the cap 62 (see FIG. 20). In this state, the cap 62 is positioned as high as it goes. In this state, the main body supports 63f and 63g are in their outermost positions. Thus, the main body supports 63f and 63g are spaced as far apart as they go. As a result, the puncture hole 12 is disposed as far away from the support holes 64 as it goes.

In FIG. 24B, the adjustment protrusion 66b of the main body 61 is latched in the adjustment hole 65b of the cap 62, and the adjustment protrusion 66c to the adjustment hole 65c. Here, the spacing between the main body support 63f and the main body support 63g is narrower than the spacing in FIG. 24A. As a result, the support holes 64 overlap the puncture hole 12.

Thus, the distance between the puncture hole 12 and the support holes 64 varies with the vertical position of the cap 62.

Furthermore, when the cap 62 is lowered as far as it will go (when not being used; see FIGS. 17A to 17C), the puncture hole 12 is blocked by the cap 62, the adjustment protrusion 66d is latched in the adjustment hole 65d, and the adjustment protrusion 66e is latched in the adjustment hole 65e. At this point, the main body supports 63f and 63g are stowed in the cap 62. Since the main body supports 63f and 63g are thus stowed, the blood testing device 60 can be carried around more easily.

If the number of adjustment protrusions and adjustment holes is increased, the spacing between the main body support 63f and the main body support 63g can be adjusted with greater flexibility.

### (3) Other Embodiments

Those members that have the same function as the members described above will be numbered the same and will not be described again.

(3-1)

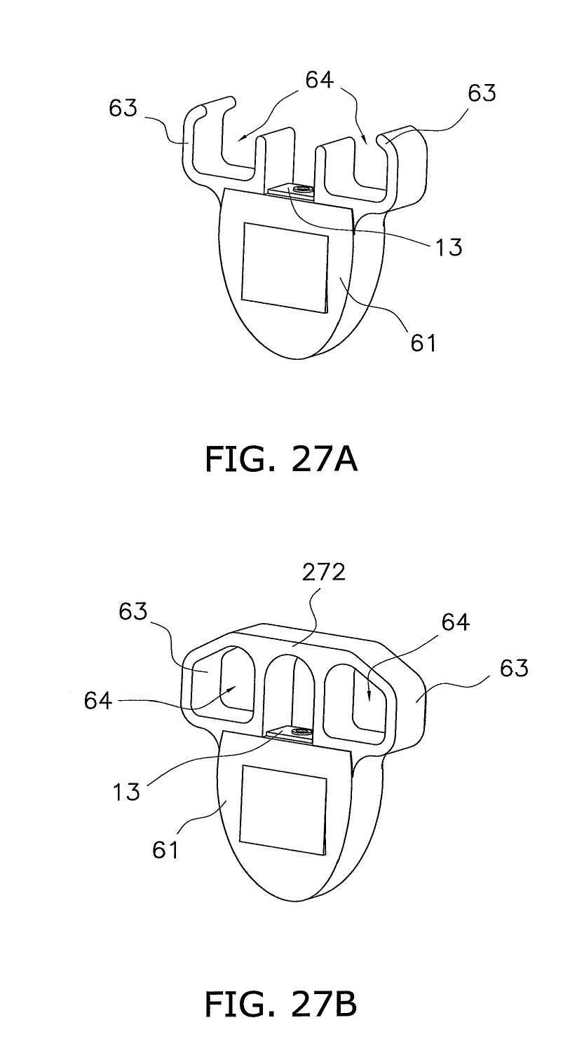

In FIG. 27A, the positions of the main body supports 63 are fixed at locations to the sides of the finger contact component 13. The support holes 64 have an open ring-shape, that is, they are U-shaped. The portion above the finger contact component 13 is open, and not covered.

The main body 61 in FIG. 27B is constituted the same as the main body 61 in FIG. 27A, except that a cover 272 is provided above the finger contact component 13 and the support holes 64 are in the form of closed rings. The cover 272 of the finger contact component 13 is not essential.

With both of the constitutions shown in FIGS. 27A and 27B, the distance between the two main body supports 63 is fixed and does not vary.

(3-2)

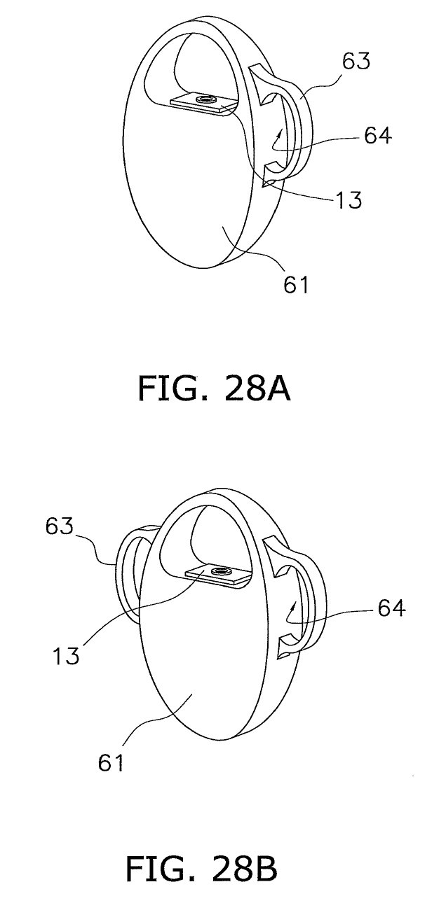

The main body 61 in FIG. 28A has one main body support 63 on one side face of the main body 61. The main body 61 in FIG. 28B has two main body supports 63 that protrude on the left and right sides of the main body 61.

With both of the aspects in FIGS. 28A and 28B, the main body supports 63 are shaped like handles. That is, the main body supports 63 are U-shaped, with both ends thereof fixed in contact with the main body 61.

With both of the aspects in FIGS. 28A and 28B, when the middle finger touches the finger contact component 13, the thumb, for example, can be inserted into the main body support 63. Specifically, a finger apart from the finger to be punctured can be inserted into the hole 64 of the main body support 63.

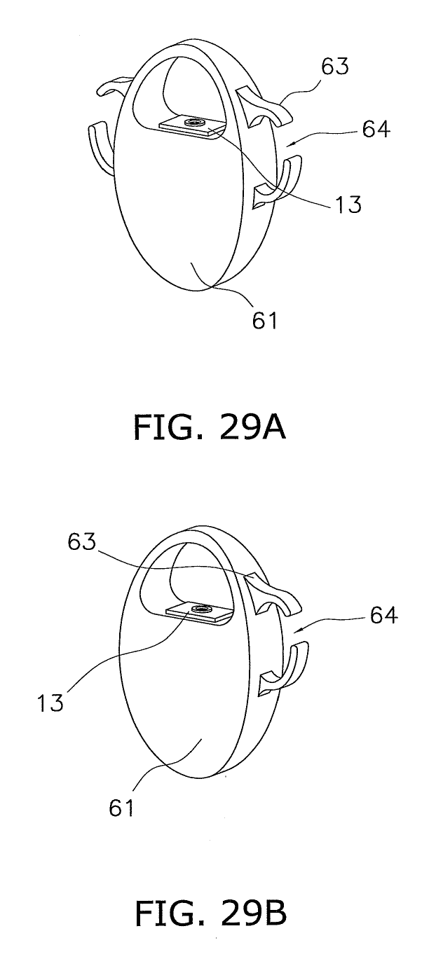

As shown in FIGS. 29A and 29B, in the aspects shown in FIGS. 28A and 28B, the main body supports 63 may have an open shape. Specifically, a single main body support 63 may be made up of two members, with a gap provided in between these two members. In other words, a single main body support 63 may be split in two.

Furthermore, a single main body support 63 may be formed from one continuous member, with just one end of the main body support 63 fixed to the main body 61, and the other end not fixed.

(3-3)

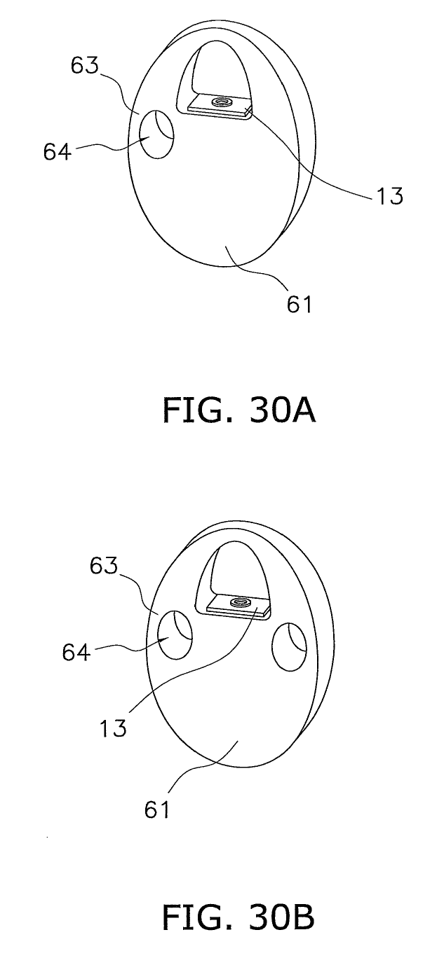

With the aspects shown in FIGS. 30A and 30B, a hole 64 is provided in the main body 61 itself. That is, part of the main body 61 is the main body support 63. With the aspect in FIG. 30A, there is just one hole 64, while there are two holes 64 in the aspect shown in FIG. 30B.

With these aspects, the main body supports 63 have a shape that does not stick out beyond the main body 61. Thus, the blood testing device can have a more compact shape.

(3-4)

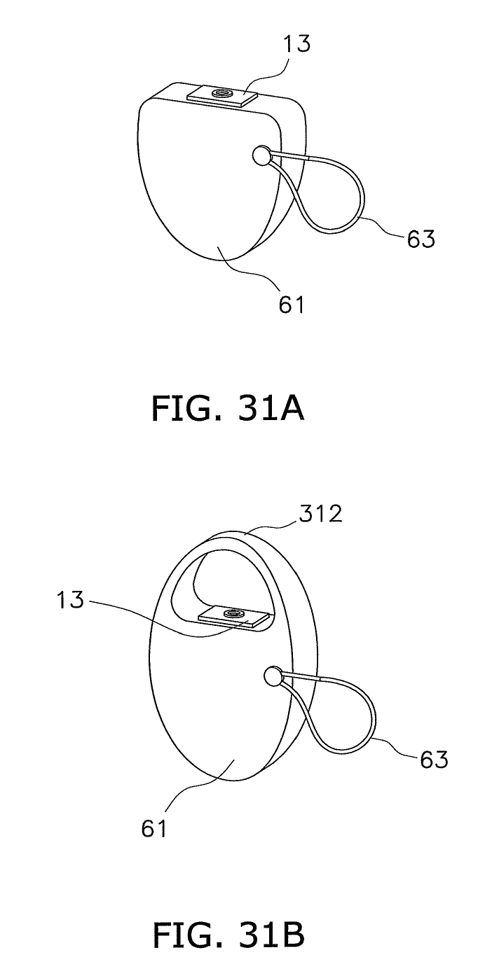

With the aspects shown in FIGS. 31A and 31B, the main body support 63 is a strap. This strap can be adjusted to size as desired.

The difference between the aspect in FIG. 31A and the aspect in FIG. 31B is whether or not a cover 312 is provided to the finger contact component 13.

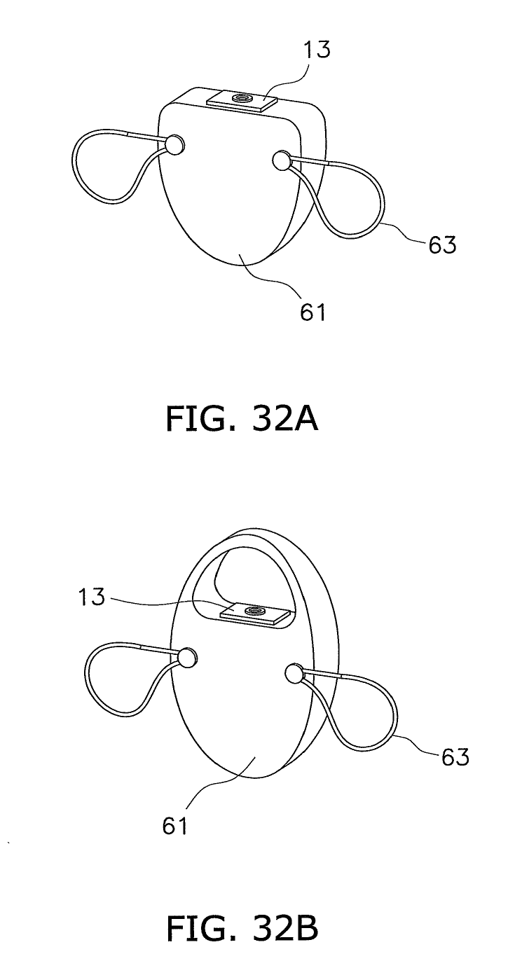

The aspects in FIGS. 32A and 32B have the same constitution as those in FIGS. 31A and 31B, except that a total of two main body supports 63 are provided to the main body 61, one on the left and one on the right.

(3-5)

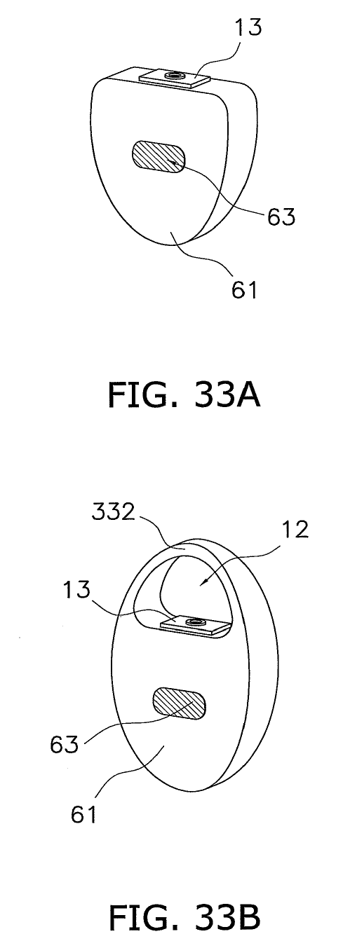

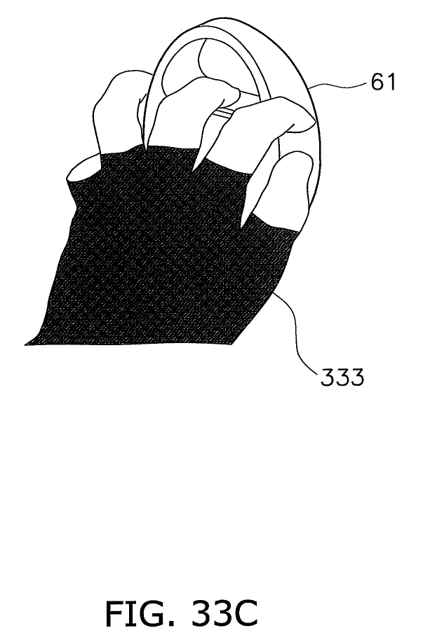

In the aspects shown in FIGS. 33A and 33B, the main body support 63 is a member that can be removed and member that can be a pair with a paired member, such as magnet, surface fastener, or hook. If the main body support 63 is a magnet, then iron, another magnet, or another such magnetic material is employed as the paired member. If the main body support 63 is a surface fastener and is the member having the hooked face, then a member having a looped face is employed as the paired member. Conversely, if the main body support 63 is a surface fastener and is the member having looped face, then a member having a hooked face is employed as the paired member. If the main body support 63 is a hook, then another hook, a prong, or a loop that can catch the first hook is employed as the paired member.

The paired member is fixed to a glove 333 (FIG. 33C) or the like from which the finger to be punctured is exposed. The paired member is mounted on the user's hand via this glove 333.

When the paired member is mounted on the user's hand, the main body 61 that is gripped in this hand is fixed to the palm. As a result, the position of the main body 61 in the user's hand is stable. This makes it less likely that the blood testing device will be dropped.

The aspect in FIG. 33B has the same constitution as the aspect in FIG. 33A, except that a cover 332 is provided to the finger contact component 13.

(3-6)

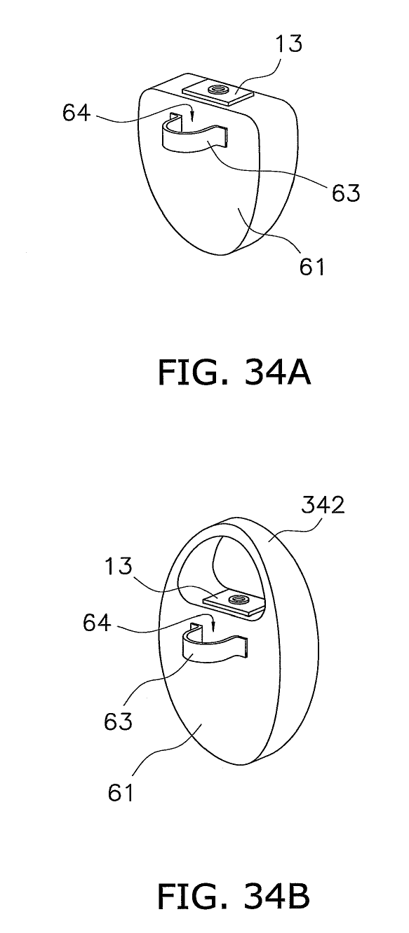

The main body supports 63 in the aspects in FIGS. 34A and 34B are provided on the rear face of the main body 61 (the opposite side from the display component 14). In these aspects, the main body supports 63 have a support hole 64 that is parallel to the planar direction of the main body 61. More specifically, the main body supports 63 are semicircular members that are fixed at both ends to the rear face of the main body 61.

The user inserts the finger to be placed on the finger contact component 13 (the finger to be punctured) in the support hole 64 of the main body support 63, and then bends this finger at a right angle to place the tip of the finger on the finger contact component 13.

With this aspect, inserting the finger (the finger to be punctured) into the main body support 63 stably fixes the position of the blood testing device with respect to the user's finger.

The aspect in FIG. 34B has the same constitution as in FIG. 34A, except that a cover 342 is provided to the finger contact component 13.

(3-7)

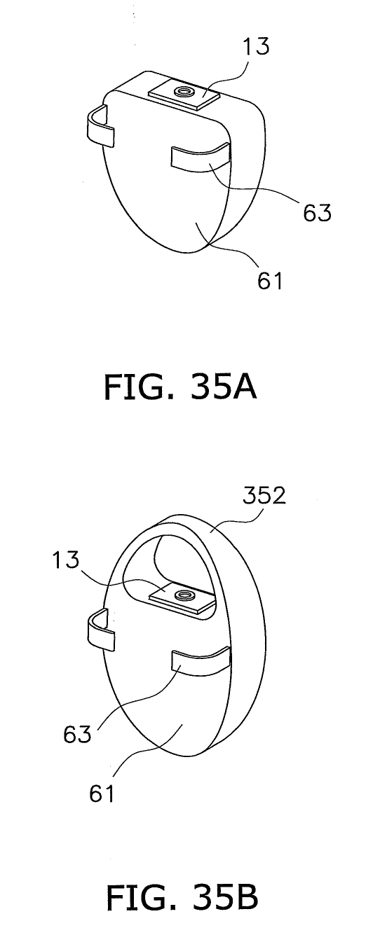

With the aspects in FIGS. 35A and 35B, the main body supports 63 are provided on the rear face of the main body 61 at locations close to the two side faces of the main body 61.

As shown in FIGS. 35A and 35B, in these aspects, the main body supports 63 are not closed, and instead have an open shape. More specifically, the main body supports 63 have a rounded L shape, and are fixed to the main body 61 at an L-shaped first end. An L-shaped second end is farther away from the main body 61.

When the user wishes to use the device, he puts his hand (the four fingers other than the thumb) between the two main body supports 63. The user then places one of the four fingers (such as the middle finger) against the finger contact component 13. At this point the main body supports 63 grasp the back of the hand and fix it to the main body 61. Rather than putting in the whole hand, a plurality of fingers (such as the index, middle, and ring fingers) may be inserted between the main body supports 63. Thus grasping the fingers or hand with the main body supports 63 allows the blood testing device to be stably held in the hand. As a result, this prevents the blood testing device from being dropped.

The blood testing device in FIG. 35B has the same constitution as in FIG. 35A, except that a cover 352 is provided to the finger contact component 13.

(3-8)

The blood testing device may be expressed as follows.

The blood testing device has the following (I) and (II).
(I) a main body, having the following (i) to (iv):
   (i) a puncture component for puncturing a finger
   (ii) a finger contact component where the finger to be punctured is placed during puncture by the puncture component
   (iii) a sensor receptacle in which a blood sensor can be mounted
   (iv) a measurement component that measures a component in the blood held in the blood sensor
(II) at least one main body support that has a first space into which a finger other than the finger to be punctured is inserted, and that is provided to the main body directly or via another member.

The various constituent elements are constituted as follows.

### (I) Main Body

### (i) Puncture Component

The puncture component punctures the skin of a finger or another part of the body, allowing blood to seep out from the user's body. This puncture component may be a needle type or a laser type. The needle type is very well known. In view of this, a laser type of puncture component will be described.

A laser puncture component specifically includes a laser rod, a flash lamp, a lens barrel, and a lens. With a compact type of device that can be held in the hand, as with the blood testing devices in the various embodiments discussed above, a solid-state laser can be used to advantage as the laser.

The laser rod is composed of a material doped with transition metal ions or rare earth ions that will serve as the laser active species, such as erbium (Er), neodymium (Nd), or holmium (Ho). The flash lamp excites the laser active species by irradiating the laser rod with light. The lens barrel efficiently converges the light of the flash lamp onto the laser rod, without letting it leak to the outside. The lens converges the light emitted from the laser rod to a specific location. Examples of solid-state lasers include ruby lasers, glass lasers, and YAG lasers.

During puncture, a YAG laser rod doped with about 50% erbium (Er) is preferable as the laser rod. The rod may be a YAG single crystal, or a YAG ceramic.

If the puncture component is a laser, a circuit for operating the laser or the like may be provided to the main body.

### (ii) Finger Contact Component

The finger contact component fixes the site to be punctured. The site to be punctured was the skin of a finger in the above embodiments, but may be a site other than a finger.

The shape of the finger contact component is preferably one that will stably hold the finger, and the material and structure are preferably such that deformation will not readily occur when pushed by a finger. The shape of the finger contact component is also preferably such that when the site put in contact with the finger contact component (the skin of a finger, etc.) is pressed toward the finger contact component by the user, this site can be compressed.

The finger contact component has an opening that allows a needle or light to pass through. This needle or light causes blood to seep out from the skin. The opening may be circular or elliptical. The skin contact part of the finger contact component is preferably not sharp, so that it will not cause the user any pain if it is pressed against a part of the body.

Also, the blood testing device may be equipped with a mechanism for applying suction to the site (skin) in contact with the finger contact component. For instance, a mechanism may be provided for applying suction to a finger from inside the opening of the finger contact component. In this case, a member that fits snugly against the skin (such as a gasket) is preferably provided to the portion that touches the skin at the opening of the finger contact component. This mechanism for applying suction to the skin squeezes out the blood that seeps from the skin.

It is preferable if the finger contact component can be easily removed from the blood testing device. This is because the user will more easily be able to clean away any blood or other soil that has adhered to the finger contact component.

In the above embodiments, the blood that seeped from the finger punctured with the puncture component is captured directly in the blood sensor. Thus, the finger contact component is disposed so as to overlap the finger contact component.

The space that includes the finger contact component may be a space that is closed off by a wall or the like, or may be a space that is open enough to allow a finger to be fixed. Specifically, a cover may be provided to the finger contact component. If the puncture component punctures the skin with a laser beam, a cover is preferably provided to the finger contact component. This is because the cover will block the laser beam.

As discussed above, when an opening is provided to the finger contact component, a cap is preferably provided. The cap will prevent dirt and so forth from getting inside the device. The main body supports may also be stowed in the cap.

(iii) Sensor Receptacle

The sensor receptacle holds a blood sensor. If the blood sensor is electrical, the sensor receptacle may be a connector.

In the above embodiments, the sensor receptacle is disposed so that a blood sensor held in the sensor receptacle will overlap the finger contact component. The sensor receptacle preferably has a structure in which the blood sensor will be held without being dropped even if the orientation of the blood testing device should change.

### (iv) Measurement Component

The measurement component processes a signal from the blood sensor and computes the concentration of a blood component, etc., on the basis of a pre-recorded table, calibration line, or the like.

### (v) Other

Besides the constitution discussed above, the main body may be constituted as a display component or the like. The measurement result is displayed on the display component. Everything but the display component and the finger contact component of the main body is surrounded by a housing.

### (II) Main Body Support

The main body support has the auxiliary role of stabilizing the main body in the user's hand during puncture, after puncture, during measurement, and so forth, so that the device is not dropped.

Basically, the main body support is preferably located on both sides of the finger contact component, and should support the main body when fingers other than the finger to be punctured are inserted. The portion of the main body support in which the fingers are inserted may be a closed space or an open space. However, the main body support preferably has a shape with which an inserted finger will not readily come loose, and which allows the main body to be held in the user's hand.

Depending on the shape and size of the main body supports, just one finger may be inserted, or a plurality of fingers may be inserted, or the whole hand may be inserted.

If the main body supports are formed from members with elasticity (plastic, etc.), they can fix the main body to the hand by their elasticity.

Also, the main body support may be constituted by a flexible material, as with a strap.

Also, the main body support may be designed to support the finger to be punctured itself. Specifically, the main body support may be disposed at a position in the main body where the finger placed against the finger contact component passes by. The main body support extends in the direction in which the finger passes, and has a hole through which the finger to be punctured passes.

Also, the main body support may have a shape like a handle with respect to the main body. The portion of this main body support in which the finger is inserted may have a closed shape or an open shape. That is, the space into which the finger is inserted may be a closed space or an open space.

Also, the main body support may be provided integrally with the main body. Specifically, a through-hole large enough for a finger to be inserted may be provided to the main body. This through-hole is used as a main body support by inserting a finger into it. Thus, the main body support may be included in the main body.

### (4) Effect of Main Body Support

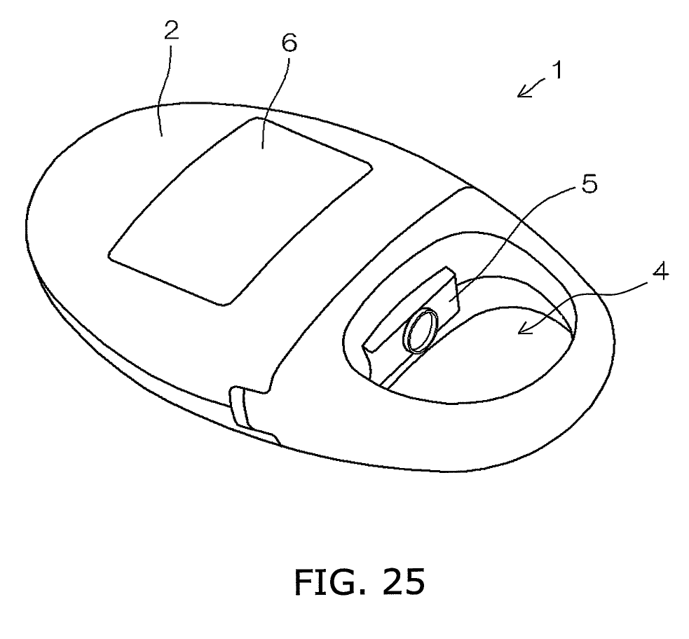

When the conventional blood testing device 1 (see FIGS. 25 and 26) is used, the user holds the blood testing device 1 by gripping the device with his hand 3. Consequently, the blood testing device 1 may slip from the palm of the hand, or may be unsteady (may jiggle).

With this blood testing device 1, there are times when the finger 3a inserted into the hole 4 comes out from the finger contact component 5 or out of the hole 4. If the finger 3a moves like this, the required amount of blood may not be captured in the blood sensor. Also, the blood testing device 1 may slip from the user's hand. Therefore, to obtain an accurate test result and to ensure the safety of the device, the blood testing device needs to be held stably with respect to the finger to be punctured.

The blood testing device in the embodiments given above has a main body support. This main body support stably fixes the main body of the blood testing device to the hand of the user. Thus, the device is prevented from being dropped, and more accurate testing is possible.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents.

Thus, the scope of the invention is not limited to the disclosed embodiments.

### General Interpretation

As used herein, the following directional terms “forward, rearward, above, downward, vertical, horizontal, below and transverse” as well as any other similar directional terms refer to those directions of a device equipped with the present invention. Accordingly, these terms, as utilized to describe aspects of the present invention, should be interpreted relative to a device equipped with the present invention.

The term “configured” as used herein to describe a component, section or part of a device includes hardware and/or software that is constructed and/or programmed to carry out the desired function.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applied to words having similar meanings such as the terms, “including,” “having,” and their derivatives. Also, the term “part,” “section,” “portion,” “member,” or “element” when used in the singular can have the dual meaning of a single part or a plurality of parts.

The terms of degree such as “substantially”, “about” and “approximately” as used herein mean a reasonable amount of deviation of the modified term such that the end result is not significantly changed.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents. Thus, the scope of the invention is not limited to the disclosed embodiments.

What is claimed is:

1. A blood testing device, comprising:
a main body, including a puncture component configured to puncture a finger, a finger contact component arranged to contact a finger to be punctured during puncture by the puncture component, a sensor receptacle arranged to be mounted with a blood sensor, and a measurement component configured to measure a concentration of a component in the blood held in the blood sensor; and
at least one main body support that has a first space into which a finger other than the finger to be punctured is inserted, and that is provided to the main body directly or via another member.

2. The blood testing device according to claim 1,
wherein the blood sensor mounted in the sensor receptacle is disposed overlapping the finger contact component.

3. The blood testing device according to claim 1,
further comprising a wall that surrounds the finger contact component,
wherein the wall is disposed so that the finger to be punctured is inserted in a second space surrounded by the wall.

4. The blood testing device according to claim 3,
wherein the puncture component comprises a solid-state laser.

5. The blood testing device according to claim 3,
further comprising a cap arranged to cover the finger contact component.

6. The blood testing device according to claim 1,
wherein the main body support comprises a closed shape that has the first space in its interior.

7. The blood testing device according to claim 1,
wherein the main body support is disposed adjacent to the finger contact component.

8. The blood testing device according to claim 1,
wherein the main body support is movable.

9. The blood testing device according to claim 8,
further comprising a wall that surrounds the finger contact component,
wherein the wall is disposed so that the finger to be punctured is inserted in a second space surrounded by the wall, and
the spacing between the main body support and the puncture wall is variable.

10. The blood testing device according to claim 5,
wherein the main body support is arranged to be stowed in the cap.

11. The blood testing device according to claim 5,
wherein the cap is arranged to slid with respect to the main body, and
the main body support moves in conjunction with the sliding of the cap.
